(19) United States
(12) Patent Application Publication
(10) Pub. No.: US 2025/0114079 A1
(43) Pub. Date: Apr. 10, 2025

(54) Ultrasound Phantom

(71) Applicants: United States Government As Represented By The Department Of Veterans, Washington, DC (US); Northwestem University, Evanston, IL (US)

(72) Inventors: Wendy M. Murray, Evanston, IL (US); Eric Perreauit, Evanston, IL (US); Cheng Sun, Evanston, IL (US); Caralyn Collins, Evanston, IL (US); Samuel Gillespie, Evanston, IL (US)

(21) Appl. No.: 18/910,230

(22) Filed: Oct. 9, 2024

Related U.S. Application Data
(60) Provisional application No. 63/588,874, filed on Oct. 9, 2023.

(51) Int. Cl. A61B 8/00; G09B 23/28

(52) U.S. Cl. CPC A61B 8/587; G09B 23/286

(57) ABSTRACT

A phantom has a structure that, when subject to a B-mode ultrasound, is configured to generate return waves that mimic return waves produced by muscle tissue when subjected to B-mode ultrasound. The phantom has a longitudinal axis. The structure includes a first material. The structure defines a plurality of regions free of the first material along a first transverse axis perpendicular to the longitudinal axis.

### CROSS-REFERENCE TO RELATED APPLICATION

This application claims priority to and the benefit of the filing date of U.S. Provisional Patent Application No. 63/588,874, filed Oct. 9, 2023, the entirety of which, including the Appendices, is hereby incorporated by reference herein.

### FIELD

This disclosure relates to phantoms for ultrasounds.

### BACKGROUND

B-mode ultrasound is a 2D imaging modality in which reflection from material interfaces and scattering from material irregularities appear as bright, echogenic spots in the resulting image frame. Muscle architectural parameters of interest to BME researchers (e.g., fascicle length), can be measured in vivo using B-mode ultrasound-based techniques. Clinical implementation of musculoskeletal B-mode ultrasound includes qualitative discrimination of muscle tissue state (e.g., healthy, paretic, spastic), diagnosis of musculoskeletal injuries (e.g., muscle tears), and guidance for injection-based therapies (e.g., botulinum neurotoxin injection).

Imaging phantoms mimic qualities of the biological tissue of interest and are used to evaluate, analyze, and tune the performance of imaging methods and train users.

### SUMMARY

Disclosed herein is phantom that has a structure that, when subject to a B-mode ultrasound, is configured to generate return waves that mimic return waves produced by muscle tissue when subjected to B-mode ultrasound. The phantom has a longitudinal axis. The structure includes a first material. The structure defines a plurality of regions free of the first material along a first transverse axis perpendicular to the longitudinal axis.

Additional advantages of the disclosed apparatuses, systems, and methods will be set forth in part in the description that follows, and in part will be obvious from the description, or may be learned by practice of the claimed invention. The advantages of the disclosed devices and systems will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

FIG. 1A shows a perspective view of a CAD model of a first exemplary phantom structure as disclosed herein. FIG. 1B shows a cross-sectional view of the first exemplary phantom of FIG. 1A.

FIG. 2A shows an ultrasound image of the phantom of FIG. 1A along a first transverse axis. FIG. 2B shows an ultrasound image of the phantom of FIG. 1A along a second transverse axis.

FIG. 3B shows a perspective view of a CAD model of a second exemplary phantom structure as disclosed herein. FIG. 3B shows a cross-sectional view of the second exemplary phantom of FIG. 3A.

FIG. 4A shows an ultrasound image of the phantom of FIG. 3A along a first transverse axis. FIG. 4B shows an ultrasound image of the phantom of FIG. 3A along a second transverse axis.

FIGS. 5A-C show long axis B-mode ultrasound snapshots, including 3D printed 5×5 bundle of 2 mm diameter rods, shown in FIG. 5A, 3D printed hexagonal honeycomb pattern, shown in FIG. 5B; and panoramic image of a healthy adult human biceps brachii, a parallel-fibered muscle, shown in FIG. 5C.

FIG. 6 shows a perspective view and a partial detail view of a third exemplary phantom structure as disclosed herein.

FIG. 7 is a schematic diagram of an exemplary setup for performing an ultrasound test.

FIGS. 8A and 8B illustrate modified pulse-echo, time-of-flight acoustic testing. FIG. 8A is a diagram illustrating the testing method used to assess bulk acoustic properties of the selected resin. Propagation path lengths of the waves reflected off the top and bottom surfaces are indicated by pairs of blue and green arrows, respectively. FIG. 8B shows a representative graph of single element transducer voltage signal (mV) vs. time (seconds), showing the waveforms of the reflected signals. The signal that reflects off the top surface of the resin sample takes a shorter time (t₁) to return compared to the time to return for the bottom reflected signal (t₂).

FIG. 9 illustrates various biceps regions of interest (ROI) and pixel intensity histograms.

FIGS. 10A-10C shows plots illustrating muscle echo intensity analysis. FIG. 10A shows echo intensity (EI) range recorded in each of the 24 images plotted as bars. Data is grouped by subject (6 images per subject) with fill denoting arm dominance. FIG. 10B shows a histogram of EI values recorded in all 72 ROI across the 24 images. FIG. 10C shows a mean standard deviation of EI within different subsets of data; within the same image, within all images of the same arm, within all images of the same subject (both arms), and overall across all images.

FIG. 11 shows a plot of echo intensity (EI) values (Mean±Standard Deviation) of rod, solid honeycomb, and lattice honeycomb designs. Grey area indicates range of EI values recorded in long axis Biceps brachii images. FIG. 11 further shows, on the left, representative high, medium, and low EI regions within muscle B-mode images, and, on the right, representative B-mode images for a lattice honeycomb phantom, a solid honeycomb phantom, and a rod phantom B-mode phantom.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

As used herein, the terms “optional” or “optionally” mean that the subsequently described event or circumstance may or may not occur, and that the description includes instances where said event or circumstance occurs and instances where it does not.

The word “or” as used herein represents disclosure of embodiments in which any one member of a particular list is provided, and, unless context dictates otherwise, also represents disclosure of alternative embodiments in which any combination of members of that list is provided.

It must be noted that as used herein and in the appended claims, the singular forms “a,” “an,” and “the” can include both single and plural references unless the context clearly dictates otherwise. Thus, for example, reference to “a support” includes aspects in which only a single support is provided, as well as aspects in which a plurality of such supports are provided.

The following description supplies specific details in order to provide a thorough understanding. Nevertheless, the skilled artisan would understand that the apparatus and associated methods of using the apparatus can be implemented and used without employing these specific details. Indeed, the apparatus and associated methods can be placed into practice by modifying the illustrated apparatus and associated methods and can be used in conjunction with any other apparatus and techniques conventionally used in the industry.

For B-mode musculoskeletal imaging, ultrasound phantoms for replicating fascicle-level image detail do not exist. 3D printing enables fast fabrication time, custom designs, and high precision for replicating small features. Clinically and scientifically relevant muscle fascicle-level imaging details can be reproduced with SLA 3D printed, photocured elastic resin.

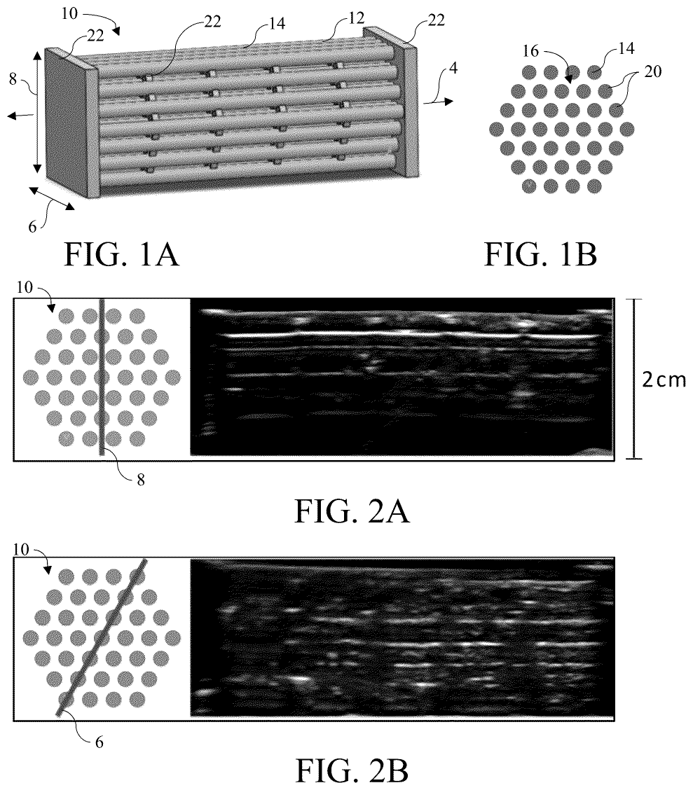

Disclosed herein, and with reference to FIGS. 1A-6, are phantoms 10 for generating outputs of an ultrasound scan that mimic return waves produced by muscle tissue when subjected to B-mode ultrasound. Accordingly, the phantom 10 can have a structure 12 that, when subject to a B-mode ultrasound, is configured to generate return waves that mimic return waves produced by muscle tissue when subjected to B-mode ultrasound.

In some aspects, the phantom 10 can have a longitudinal axis 4. The structure 12 can comprise a first material 14. The structure 12 can define a plurality of regions 16 free of the first material 14 along a first transverse axis 6 perpendicular to the longitudinal axis 4.

Referring to FIGS. 1A-2B, in some optional aspects, the structure 12 can comprise a plurality of rods 20 that are elongated along the longitudinal axis 4. The structure 12 can further comprise at least one support 22 that spaces the plurality of rods along the first transverse axis. The plurality of regions 16 free of the first material 14 can be defined by spaces between the plurality of rods 20. The at least one support 22 can further space the plurality of rods 20 along a second transverse axis 8 that is perpendicular to the longitudinal axis 4 and the first transverse axis 6.

In some aspects, the plurality of rods 20 can be arranged in a honeycomb pattern. In some aspects, the plurality of rods can have a diameter from about 1 mm to about 4 mm, or from about 1 mm to about 3 mm, or about 2 mm. Optionally, the plurality of rods 20 can be cylindrical. In other aspects, the plurality of rods 20 can be square, rectangular, hexagonal, or any suitable shape.

In some aspects, sequential rods of the plurality of rods 20 can be evenly spaced from each other. For example, the two or more of the plurality of rods can be aligned along at least one transverse axis (e.g., the first transverse axis 6 or the second transverse axis 8). Sequential rods of the two or more rods 20 aligned along said a given transverse axis can optionally be equally spaced (e.g., center-to-center). In other aspects, the sequential rods aligned along a given transverse axis can be unevenly spaced.

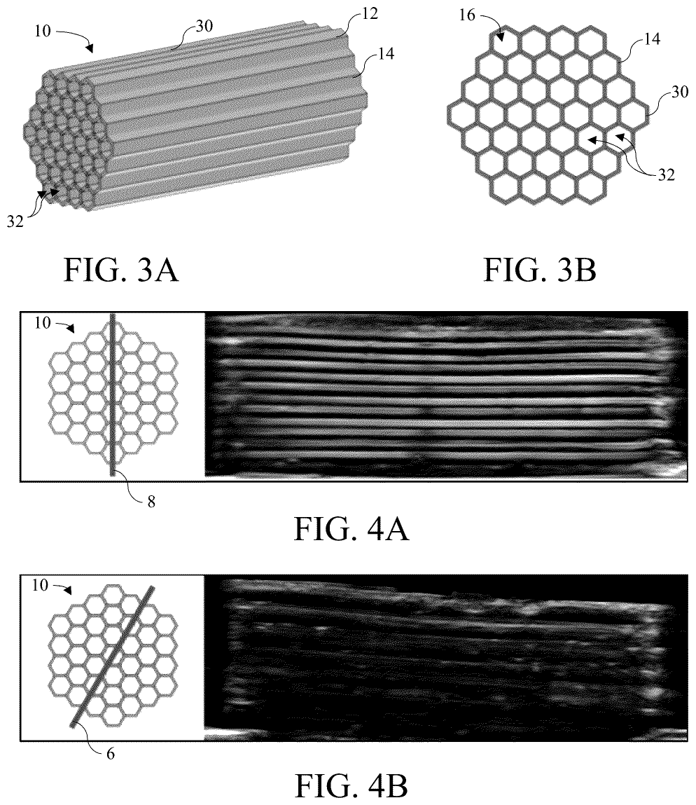

In some aspects, and with reference to FIGS. 3A-4B, the structure 12 can comprise a body 30 defining a plurality of longitudinally extending passages 32 that define the plurality of regions 16 free of the first material 14. For example, in some aspects, the plurality of longitudinally extending passages 32 can be hexagonal in cross sections perpendicular to the longitudinal axis 4. In other aspects, the longitudinally extending passages 32 can be round (e.g., circular) square, rectangular, or any suitable shape.

The plurality of longitudinally extending passages 32 can have an inscribed diameter from about 1 mm to about 4 mm. Optionally, the plurality of longitudinally extending passages 32 can have an inscribed diameter from about 1 mm to about 3 mm, or about 2 mm).

Referring also to FIG. 6, the structure 12 can comprise walls 34 defining the longitudinally extending passages 32. The structure 12 can further defines transverse openings 36 extending through the walls 32 of the longitudinally extending passages. For example, the transverse openings of adjacent walls of adjacent passages can be axially offset from each other. Accordingly, the structure can have a latticed profile.

In some aspects, the plurality of regions 16 free of the first material 14 can be full of a fluid (e.g., gas or liquid). For example, the plurality of regions 16 free of the first material 14 can be filled with water. In other aspects, the plurality of regions 16 free of the first material 14 can be full of a second solid material that is different from the first material 14.

It is contemplated that the phantom 10 does not have an internal structure that physically resembles muscle. For example, muscle can comprise closely positioned fascicle having essentially no air gaps therebetween. The phantom can cause an ultrasound to generate an output that mimics the muscle tissue even though the phantom does not have the actual structure of the muscle tissue itself.

In some aspects, the phantom 10 can have a length from 20 mm to 100 mm, (e.g., about 50 mm).

In various aspects, the first material 14 can comprise photocured elastic resin. In some optional aspects, the first material 14 can have a hardness from about 40 to about 60 on the shore A durometer scale. For example, the first material 14 can have a hardness from about 45 to about 55, or about 50 on the shore A durometer scale.

In some aspects, the phantom 10 can be formed by 3D printing (e.g., via stereolithography (SLA)).

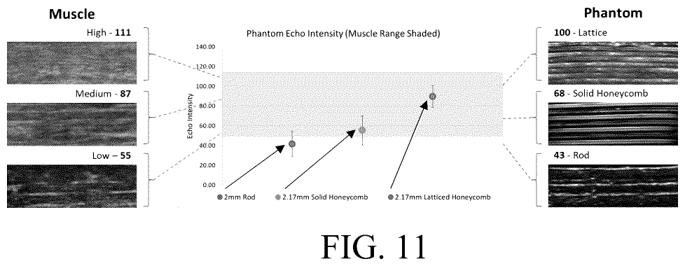

Referring to FIG. 11, echo intensity can be the metric used to indicate the extent to which the phantom mimics muscle tissue for purposes of B-mode ultrasound imaging. Echo intensity is a measure of the ability of a tissue or structure to reflect or transmit sound waves. In-vivo ultrasound images of muscle structure are characterized by hypoechoic (dark) muscle fascicles surrounded by hyperechoic (bright) fibrous and adipose tissues.

Echo intensity of the B-mode images collected from multiple phantoms of different designs were compared to 24 Biceps brachii images acquired in 8 arms (both arms of four different people). Data from the phantoms and from the living human subjects were collected with the same ultrasound machine. For each of images (in vivo images of human biceps and images of the phantom) multiple regions of interest were sampled, converted to DICOM format, in 8-bit grayscale. Echo intensity within a selected region of interest was defined as the average pixel intensity from 0 to 255 arbitrary units (AU).

Echo intensity values (mean±SD) measured in phantom B-mode images of three different phantom structures were 42±13 AU, 55±15 AU and 90±11 AU, respectively (see FIG. 11).

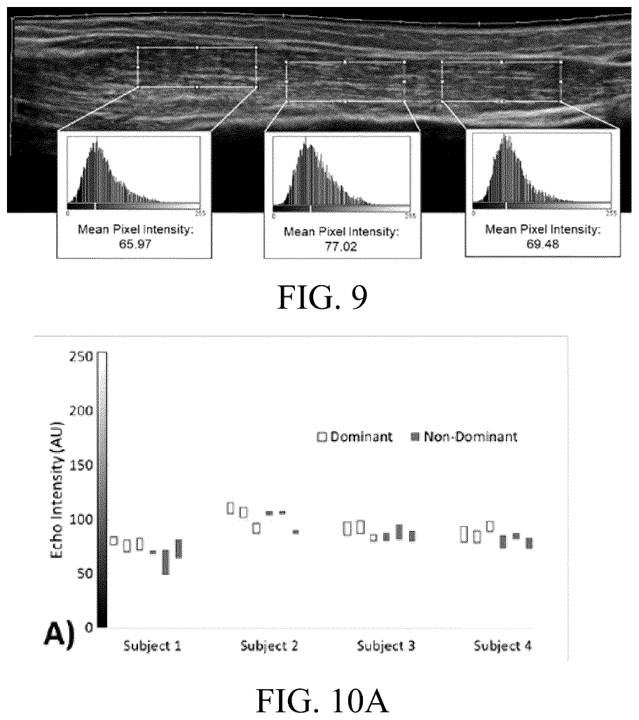

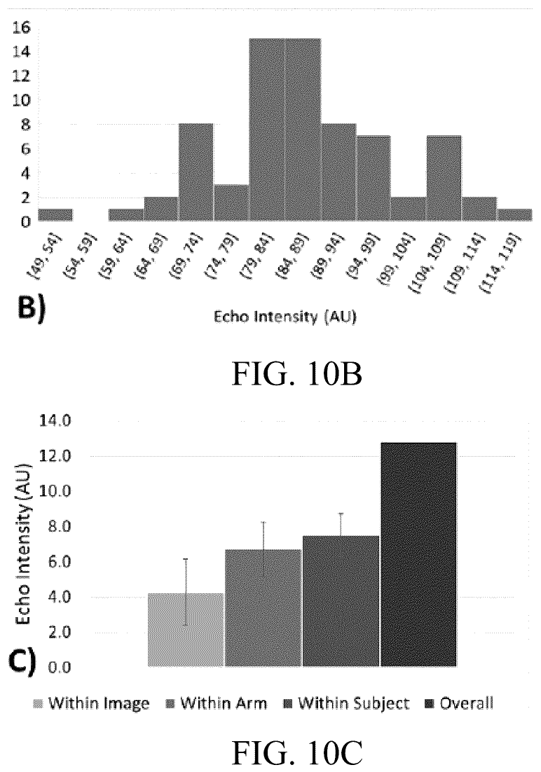

The overall mean Echo Intensity value across all 72 ROIs in all images from the human biceps brachii was 87±13 AU, shown in FIGS. 9-10C. The mean Echo Intensity values from all 72 ROIs had an approximately normal distribution, spanning a range of 49 AU to 116 AU (FIG. 10B). The mean standard deviation of Echo Intensity values within data subsets were; 4.3 AU within individual images, 6.7 AU within images of the same muscle, 7.5 AU within both arms of the same subject, and 12.8 AU across all data (FIG. 10C).

Accordingly, it is contemplated that the phantom 10 can provide an echo intensity of from about 30 AU to about 120 AU, or from about 50 AU to about 110 AU, or from about 30 AU to about 50 AU, or from about 40 AU to about 70 AU, or from about 75 AU to about 105 AU. Further, in various optional aspects, the phantom can provide an echo intensity standard deviation from about 10 to about 15 AU. In exemplary aspects, the phantom 10 can provide an echo intensity of from about 35 AU to about 50 AU and a standard deviation of about 10 to about 20. In further exemplary aspects, the phantom 10 can provide an echo intensity from about 50 to about 60 AU and a standard deviation of about 10 to about 20. In additional exemplary aspects, the phantom 10 can provide an echo intensity from about 60 to about 70 AU and a standard deviation of about 10 to about 20. In additional exemplary aspects, the phantom 10 can provide an echo intensity from about 70 to about 80 AU and a standard deviation of about 10 to about 20. In additional exemplary aspects, the phantom 10 can provide an echo intensity from about 90 to about 110 AU and a standard deviation of about 10 to about 20.

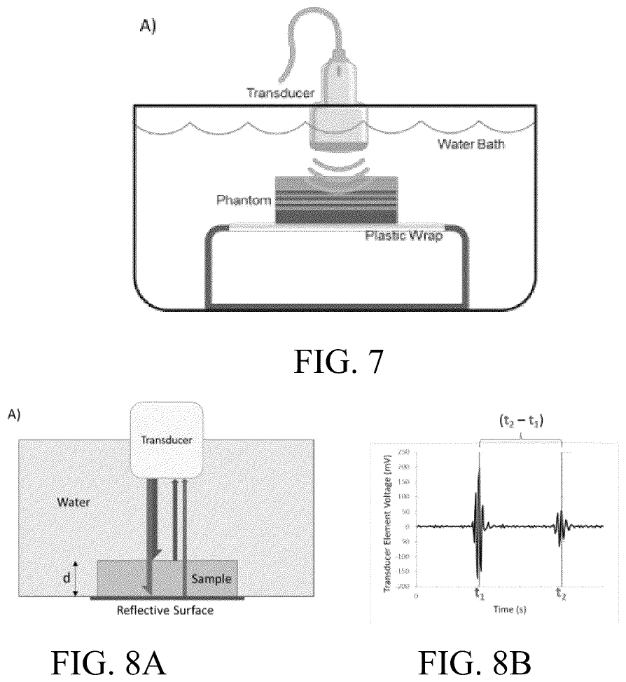

Referring to FIG. 7, a method of using the phantom 10 can comprise immersing the phantom in water. The phantom can be positioned against (and, optionally, coupled to) a reflective material. The reflective material can be, for example, a graphite wafer. A transducer can be oriented perpendicularly or generally perpendicularly to the longitudinal axis of the phantom. For example, the transducer can be oriented to capture return signals along the first transverse axis 6 or second transverse axis 8 (FIGS. 2A-2B, 4A-4B). A computing device can be configured to receive and process data associated with the return signals. For example, the computing device can translate data from the return signals into an image (e.g., a black-and-white or grayscale image). Optionally, the computing device can be that of a conventional ultrasound instrument.

### Example 1

Materials and Methods: FormLabs Elastic 50A resin was selected as a print material because it is the softest FormLabs resin available. Solidworks 3D CAD software was used to create digital models of print designs consistent with parallel-fibered muscle fascicles. The resulting files were imported into and submitted to the printer through PreForm, FormLabs' 3D printing setup, management, and monitoring software. Multiple sample designs were printed at once due to the relatively large available print volume of the Form3 printer (145×145×185 mm³). Upon print completion, the samples were post-processed with Form Wash and FormCure as specified by FormLabs. B-mode ultrasound imaging of 3D printed samples was performed post-cure with an Echo Blaster 128 portable ultrasound system while the samples were submerged and supported in a water bath.

Results and Discussion: Printed samples comprising 2 mm diameter rods show echogenic surface interfaces when subjected to B-mode ultrasound imaging. When imaged along their long axis, the samples resulted in a pattern of hyper-and hypo-echogenic lines contrasting the printed ‘fascicle’ rods and the surrounding water. An alternative approach of printing the honeycomb-shaped connective tissue between hollow ‘fascicles’ showed similar echogenic surface interfaces and contrast for ‘fascicle’ differentiation.

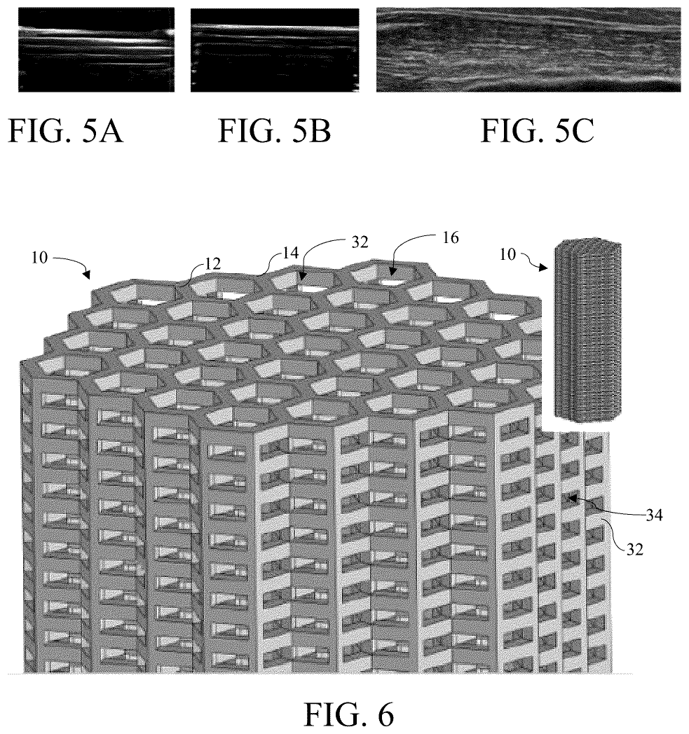

FIGS. 5A-C show long axis B-mode ultrasound snapshots, including 3D printed 5×5 bundle of 2 mm diameter rods, shown in FIG. 5A, 3D printed hexagonal honeycomb pattern, shown in FIG. 5B; and panoramic image of a healthy adult human biceps brachii, a parallel-fibered muscle, shown in FIG. 5C.

Conclusions: SLA 3D printing with soft, elastic resin shows promise for creating muscle ultrasound phantoms with fascicle-level resolution. Current samples demonstrate hyper-/hypo-echogenicity contrast between muscle fascicles and connective tissue which is a critical component of mimicking the B-mode ultrasound imaging qualities of muscle tissue. The flexibility of 3D printing allows for a wide range of phantom designs and should enable the translation of promising B-mode ultrasound imaging qualities into muscle-like phantom shapes and sizes.

### Example 2

OBJECTIVES: Imaging phantoms for training and validation are vital to improving the performance and adoption of ultrasound imaging modalities in clinical and pre-clinical applications, and the goal of this study was to assess the viability of 3D printed muscle ultrasound phantoms to meet this need.

METHODS: We used a soft stereolithography resin to 3D print phantoms that mimicked the fascicle-and perimysium-scale structure of skeletal muscle and compared the long axis B-mode imaging quality and pattern of the phantom to that of healthy, adult Biceps brachii. We used a pulse-echo, time-of-flight method to measure the acoustic impedance of the resin for comparison to skeletal muscle and common soft tissue mimicking materials. We analyzed the echo intensity (EI) of muscle images to establish a physiological range and compared the EI of different phantom designs to assess the ability to control.

RESULTS: A linear, striated hyper-/hypo-echoic B-mode imaging pattern mimicking long axis Biceps brachii muscle images was achieved with two 3D structure paradigms, rod and honeycomb. Acoustic impedance of Elastic 50A resin is higher than skeletal muscle in bulk, but appears suitable for use in a 3D structured phantom. EI measured in the Biceps images were found to vary both within and across images with an overall mean±SD of 87±13 AU. EI measured in honeycomb phantoms (55±15 AU) was higher than in rod phantoms (42±13 AU), and a latticed honeycomb further increased EI (90±11 AU).

CONCLUSIONS: This study serves as proof-of-concept for soft, 3D printed phantoms that replicate the characteristic muscle ultrasound imaging pattern with the ability to tune clinically relevant EI values via structural design.

### Introduction

Due to the structure-function relationship of skeletal muscle 1-4, measurement of macroscopic structural parameters (e.g., volume, cross-sectional area, fascicle length, pennation angle) provides fundamental information for the assessment of muscle function. In addition, skeletal muscle composition, characterized by the degree of adipose and/or fibrous tissue infiltration into muscle, provides an evaluation of muscle quality in healthy, diseased, and aging populations⁵⁻⁸. Magnetic resonance imaging (MRI) and computed tomography (CT) are the gold standard modalities for in-vivo imaging of skeletal muscle structure and composition⁹ but each suffers from limited accessibility 10,¹¹ For CT, exposure to ionizing radiation is an additional consideration^{12,13}. B-mode ultrasonography is an accessible, safe, portable, non-invasive, and cost-effective alternative to evaluate skeletal muscle structure and composition¹⁴.

In-vivo ultrasound images of muscle structure are characterized by hypoechoic muscle fascicles surrounded by hyperechoic fibrous and adipose tissues¹⁵. The parallel arrangement of muscle fascicles results in a striped appearance in longitudinal images, and a spotted, “starry night” appearance in transverse images¹⁶. Clinically, these characteristic B-mode ultrasound imaging patterns allow for qualitative evaluations of muscle tissue (e.g., healthy, aging, spastic)¹⁷, diagnosis of musculoskeletal injury^{15,16}, and guidance for biopsies and injection-based therapy^{18,19}. The 4-point Heckmatt grading scale is a qualitative clinical assessment in which spastic muscle is graded visually based on its relative echo intensity (EI) compared to bone or fat; higher scores indicate a brighter image, suggesting reduced contractile material. When used in the clinical treatment of motor impairments for stroke, a higher Heckmatt score reflects a reduction in the potential therapeutic effect of Botox injection^{17,20}. EI can be quantified by measuring the mean pixel intensity within a specified region of interest; the interpretation of higher EI as an indication of increased non-contractile tissue infiltration within the muscle is supported by MRI, CT, and histological evidence^{6,14,21-24}.

There is a critical, unmet need to improve validation methods for both clinical and pre-clinical research applications of musculoskeletal ultrasound. Improving validation of quantitative measures of muscle structural parameters using B-mode ultrasound commonly reported in research studies (e.g. fascicle length, pennation angle) has been identified as an important objective for the field²⁵. Clinically, the use of ultrasound is highly user-dependent; a lack of standardized training limits the reliability of ultrasound assessments^{26,27}. Imaging phantoms provide a useful approach to evaluate, tune, and improve performance of any medical imaging modality. Current ultrasound imaging phantoms are comprised of molded tissue mimicking materials which replicate the bulk acoustic properties of human soft tissues²⁸. The heterogeneous and anisotropic nature of skeletal muscle requires a phantom that can replicate not only bulk acoustic properties, but muscle's characteristic imaging patterns that are dependent upon imaging plane, muscle structure and composition.

Development of muscle-like, B-mode imaging phantoms would serve as a first step toward addressing validation needs, as well as reliability and standardization of training in both research and clinical settings. We hypothesized that soft, 3D printed phantoms could mimic the structural geometry and ultrasound imaging properties of skeletal muscle, while also enabling rapid prototyping and customization of phantom design. We tested this hypothesis by designing and printing 2 phantom structures, one in which the printed structure mimicked the 3D geometry and dimensions of skeletal muscle fascicles (rod) and an inverted design intended to mimic the perimysium (honeycomb). We first qualitatively compared the B-mode imaging pattern of each design to in-vivo muscle images and measured the bulk acoustic properties (impedance and speed of sound) of the 3D printed resin for insight into the echogenicity of our phantoms and comparison to skeletal muscle and common tissue mimicking materials. We also quantified the echo intensity of B-mode images of our phantoms and directly compared to muscle images. Finally, we assessed our ability to adjust echo intensity of the phantom images via introduction of latticing to the honeycomb structure.

### Materials

Stercolithography (SLA) printing was chosen for its capacity to 3D print high fidelity small features on the scale of muscle fascicles (100μ minimum feature size). FormLabs Elastic 50A resin (FormLabs, Somerville, MA) was selected as the softest commercially available SLA printer resin with a print area (145 mm×145 mm×185 mm) that would allow for printing structures on the scale of whole human skeletal muscles. 3D models of muscle fascicle structure developed in SolidWorks 3D CAD software (Dassault Systemes, Vélizy-villacoublay, France) were printed using the FormLabs Form3B SLA 3D printer. B-mode images of printed phantoms were recorded with a SIEMENS ACUSON S2000 ultrasound instrument using an 18L6 linear transducer. Long axis extended field-of-view (cFOV) ultrasound images of the Biceps brachii muscles of both arms of 4 healthy adult subjects acquired using the same ultrasound machine, obtained from a previous study²⁹, were analyzed to compare the B-mode imaging pattern and brightness of phantom and in-vivo muscle images. Acoustic properties of the resin were quantified using a single GE 9LD wide band linear transducer with the Verasonics Vantage 256 Ultrasound Imaging System (Verasonics, Kirkland, WA).

### Methods

### 3D Model Design

Human skeletal muscle fascicles are composed of 20-80 muscle fibers30,³¹, each 20-100 μm in diameter32,³³, grouped together and encapsulated by the perimysium, providing an estimated fascicle diameter range of 0.4-8 mm. Using Solidworks 3D CAD software, we designed models to mimic the tissue structure of parallel fibered muscles on the fascicle and perimysium scale (FIGS. 1A-1B, 3A-3B). The rod design directly represents fascicles as solid, 50 mm long, 2 mm diameter printed fibers with unfilled space between them representing the perimysium. The honeycomb design inverts the rod design, with fascicles represented as unfilled, hexagonal pores of 2.17 mm inscribed diameter in a 50 mm long honeycomb-like printed structure representing the perimysium.

### 3D Printing and Postprocessing

CAD models were imported as .STL files into FormLabs' PreForm software for printing, and external supports were autogenerated by the software to enhance print stability. Using PreForm, the prepared models were then submitted to the printer. A Form3B SLA 3D printer was used to print the models using default settings for the Elastic 50A resin with a layer thickness of 100 μm. In SLA 3D printing, the print platform, and subsequent cured layers, are lowered into a photocurable resin, and a rastering laser selectively cures a region as defined by the CAD model to form the next layer. Upon print completion, external supports were removed and models were washed in isopropanol to remove uncured resin. The resulting structures were air dried and finished by curing under 405 nm UV light at 60° C. in Formlabs' Form Cure mechanism for 20 minutes according to suggested curing conditions from Formlabs.

### Ultrasound Imaging

To evaluate the image quality of the 3D printed muscle phantoms, B-mode images were recorded with the Siemens Acuson 2000. Imaging parameters were set to the musculoskeletal exam default with 0 dB gain and a frequency of 7 MHz. Phantoms were submerged in water and images were recorded with the transducer in a vertical, long axis imaging plane (FIG. 7).

### Acoustic Testing

In order to test whether the acoustic properties of the selected resin were appropriate for skeletal muscle, we developed a modified pulse-echo, time-of-flight technique (FIGS. 8A-8B) based upon previously reported methods used to quantify Speed of Sound (SoS) in TMMs³⁴⁻³⁶. Flat, rectangular samples (7 cm×3 cm) with two different thicknesses (either 4 mm or 5 mm) were designed, printed, and post-processed. The resulting sample thickness (c.f., d, FIG. 8A) was confirmed before testing. In each measurement, a sample was secured to a reflective graphite wafer with the transducer fixed vertically to ensure signal propagation perpendicular to the plane of the sample and bottom surface. The transducer, sample, and reflective surface setup were immersed in a water bath for testing. A single transducer element was programmed to produce a sinusoid burst waveform with center frequency of 5.2083 MHz. The same transducer element remained active to receive the waveform reflections from the top surface of the sample and the bottom surface to which it was secured. The time point of the maximum absolute value of the transducer element voltage within the first reflected waveform (cf., t1, FIG. 8B) was subtracted from the corresponding time point of the second reflected waveform signal (c.f., 12, FIG. 8B) to determine the time-of-flight difference between the waves that reflected off the sample surface and those transmitted through the sample and reflected off the bottom surface. SoS was calculated as:

$$S o S = 2 d / ( {t}_{2} - {t}_{1} ) \qquad ( 1 )$$

Specific acoustic impedance, z, of the resin was then calculated as the product of sample SoS and density, ρ:

$$z = S oSx ρ \qquad ( 2 )$$

Sample density was determined separately by measuring the mass and volume of printed and cured cubes of resin. For method validation, measurement of the SoS in water with no sample present resulted in a value of 1485±3 ms⁻¹ which agrees with the known value of 1482 ms⁻¹ at 20° C.^{37,38}

### Echo Intensity Analysis

To assess the echo intensity (EI) levels of B-mode images of our 3D printed muscle phantoms relative to muscle images, EI analysis was performed of phantom and muscle images collected with the same ultrasound machine. All image analysis was completed with ImageJ (NIH, Bethesda, MD). To provide an in-vivo muscle EI baseline and relevant range, a total of 24 eFOV long axis Biceps brachii images were analyzed, comprised of three images acquired in both arms of four subjects²⁹. Three rectangular regions of interest (ROIs) were defined within a single muscle in each image, characterizing image brightness in the distal, middle, and proximal regions of the image (FIG. 9). Four long axis phantom images were analyzed for each 3D printed phantom. Three rectangular ROIs were defined within each image, representing shallow, middle, and deep regions of the image. In all cases, B-mode ultrasound images saved in DICOM format were converted to 8-bit grayscale. EI within a selected ROI was defined as the average pixel intensity from 0 to 255 arbitrary units (AU).

Referring to FIG. 9, a long axis extended field-of-view Biceps brachii image with (left to right) Distal, Middle, and Proximal regions of interest are outlined and corresponding grayscale pixel intensity histograms are included. Echo intensity within each ROI is the mean pixel intensity value shown below the histogram.

### Echo Intensity Control

Given the clinical importance of alterations in echo intensity for assessment of muscle quality^{17,} 20, 39, we evaluated the ability to adjust echo intensity of B-mode images of a phantom made from a specific resin via altering the 3D printed structure. To accomplish this, we introduced latticing to the honeycomb structure, with the intention of increasing ultrasound penetration and altering the reflection pattern of the resulting phantom. For this proof of concept analysis, we created a lattice design which introduces 1.25 mm×0.5 mm gaps spaced 0.5 mm apart in the long axis of the walls of each pore of the solid honeycomb design (FIG. 3A).

### B-mode Pattern Replication

A linear, striated, alternating hyper-and hypo-echoic pattern mimicking long axis muscle B-mode images was achieved with both the 3D structure that mimicked the 3D geometry and dimensions of skeletal muscle fascicles (rod) and inverted design intended to mimic the perimysium (honeycomb). The linearly patterned geometry of both printed phantoms results in a highly regular contrast pattern that was deemed qualitatively successful (FIGS. 2A-2B, 4A-4B), despite its regularity, which lacks the natural variation found in muscle images.

### Acoustic Properties

The speed of sound within and acoustic impedance of Elastic 50A are higher than skeletal muscle and common bulk soft tissue mimicking materials. The measured ultrasonic speed of sound and calculated acoustic impedance of Elastic 50A are, respectively, 8% and 13% greater than the values reported for human skeletal muscle (Table. 1). For comparison, the SoS in Agarose, PAA, and PVA, respectively, are −5%, 0%, and-1% different and the corresponding impedance values are −5%, 4%, and 5% different than skeletal muscle.

TABLE 1

| | Speed of Sound | SoS % | Impedance | Impedance % | Frequency |
|---|---|---|---|---|---|
| Material | (m s⁻¹) | Difference | (MRayl) | Difference | (MHz) |
| Elastic 50A | 1710 ± 30 | 8% | 1.87 ± 0.03 | 13% | 5.2083 |
| ^{a}Skeletal | 1580 | — | 1.66 | — | — |
| Muscle40, 41 | | | | | |
| Agarose 2%⁴² | 1500 ± 30 | −5% | 1.57 ± 0.08 | −5% | 5 |
| PAA 10%⁴² | 1580 ± 50 | 0% | 1.73 ± 0.08 | 4% | 5 |
| PVA⁴² | 1570 ± 20 | −1% | 1.74 ± 0.08 | 5% | 5 |

### Muscle Echo Intensity Analysis

Within the 24 long axis eFOV Biceps brachii images we analyzed, there was echo intensity (EI) variation across all subsets of data, even within the same muscle in a single image. The overall mean EI value across all 72 ROIs in all images was 87±13 AU. The mean EI values from all 72 ROIs had an approximately normal distribution, spanning a range of 49 AU to 116AU (FIG. 10B). The mean standard deviation of EI values within data subsets were; 4.3 AU within individual images, 6.7 AU within images of the same muscle, 7.5 AU within both arms of the same subject, and 12.8 AU across all data (FIG. 10C).

### Phantom Echo Intensity Control

In phantoms printed using the same resin, B-mode echo intensity levels varied depending on the 3D structure. Echo intensity measured in the solid honeycomb was higher than the rod, and the lattice honeycomb design further increased the EI and most closely replicated the overall mean EI measured in long axis Biceps brachii images. Echo intensity values (mean±SD) measured in phantom B-mode images of the rod, solid honeycomb, and lattice honeycomb designs were 42±13 AU, 55±15 AU and 90±11 AU, respectively (FIG. 9).

### Discussion

In-vivo muscle ultrasound is increasingly used to study muscle structure and composition in clinical and pre-clinical settings, but there remains a critical need for validation and training methods to improve adoption and reliability of ultrasound assessments. Our goal was to investigate the feasibility of using a soft, elastomeric resin to create 3D printed phantoms that mimic the acoustic and ultrasound imaging qualities of human skeletal muscle. We designed 3D structures mimicking muscle fascicles and perimysium and assessed their B-mode imaging pattern compared to in-vivo muscle images. We then tested the speed of sound and acoustic impedance of the soft, clastomeric SLA resin for insight into the echogenicity of our phantoms. Finally, we analyzed the echo intensity of our phantoms and introduced latticing to our design to demonstrate our ability to control phantom echo intensity through structural changes.

The 3D printed phantoms based upon skeletal muscle fascicle and perimysium dimensions successfully demonstrated an ability to mimic the striped hyper-and hypo-echoic pattern of in-vivo images. The hyperechoic lines in the phantoms are created by reflection from the material surfaces of the fibers and pores within the rod and honeycomb structures, respectively, and the hypoechoic spacing corresponded to the internal gaps within the phantom that were filled with water when submerged during imaging. A comparison of the hyperechoic line spacing in the Biceps brachii images, measured as low as 0.2 mm, and the phantom images, measured no lower than 0.5 mm as expected given design dimensions, indicates a need to reduce the printed structure dimensions to match in-vivo muscle images more closely. Initial efforts to reduce the fiber and pore diameters to match the dimensions of muscle imaging patterns identified fiber print stability (rod) and pore resin drainage (honeycomb) as issues that prevented designs with diameters below ˜2 mm. Even with 2 mm fibers, rod phantoms required the use of supports between fibers to maintain stability during printing. The introduction of latticing to the honeycomb holds promise for not only echo intensity tuning, but also to allow for increased resin drainage and further pore diameter reduction. Assessment of the variability of hyperechoic spacing in different muscles and subjects would help to set a dimensional goal for pore/fiber diameter and define the design parameter changes necessary for muscle-like phantoms to mimic specific muscles.

The measured speed of sound and acoustic impedance of Elastic 50A resin was higher than literature values for skeletal muscle and other commonly adopted soft tissue mimicking materials. These soft tissue mimicking materials are prepared via molding and therefore lack the capacity to control internal 3D structure, a major advantage of 3D printing^{28,43}. As opposed to molded materials which mimic the bulk acoustic properties of skeletal muscle, our phantoms introduce a 3D structure. As such, the properties of the resin alone do not accurately represent the bulk properties of a printed phantom. The higher acoustic impedance of the printed resin places surface reflection as the predominant mode of echogenic contrast within the phantom, as opposed to the volumetric scattering of muscle tissue and bulk tissue mimicking materials. In combination with high fidelity printing of fascicle-scale 3D structures, this mode of reflection can be harnessed to mimic the anisotropic nature and imaging pattern of skeletal muscle. Further work needs to be done to characterize the bulk acoustic properties of 3D patterned phantoms immersed in water or backfilled with a soft material.

Due to the periodic geometry of the printed phantoms, image quality and brightness were dependent upon the imaging plane. All images shown in the study and used for echo intensity analysis were recorded in the vertical, long axis plane of the phantoms which provided the highest quality images and greatest hyper-and hypo-echoic contrast. The high user dependence of clinical muscle imaging, which involves determination of an appropriate imaging plane, serves as an analogy and potential application of this angle dependent imaging in training new clinicians. The periodic geometry of the phantoms also creates the unnaturally regular B-mode imaging pattern because the hyperechoic lines are generated by perfectly straight, regularly spaced material surfaces. Incorporating realistic muscle cross-sectional patterns and fascicle paths via translation of in-vivo muscle images⁴⁴⁻⁴⁶ or muscle finite element modelling strategies⁴⁷ to the CAD models could eliminate the highly periodic geometry and create imaging phantoms that more closely resemble the natural variation found in muscle.

Echo intensity is a useful and accessible tool for quantifying muscle quality via ultrasound, but there are many confounding factors that limit comparisons across different instruments, imaging parameters, and subjects. This variation found within and across in-vivo muscle B-mode images, and the fact that echo intensity differences as opposed to exact values are important for clinical evaluation, indicates that there is no correct, “gold-standard” echo intensity value that a muscle phantom should aim to replicate. It is more important that a phantom design has the tunability to replicate the range of echo intensity values that are likely to be found in muscle images from diverse subject groups (e.g., healthy, strained, aging, spastic). The introduction of latticing as a structural control of echo intensity and the ability to rapidly prototype and adjust design parameters is a major benefit of 3D printed phantoms. At the measurement frequency of 5.2083 MHz, the wavelength of ultrasound waves is ˜0.3 mm within water and resin mediums, which is only slightly shorter than the width of the smallest lattice features, 0.5 mm. As the feature size of lattice structures approaches and drops below the wavelength of the ultrasound waves, reflection will more closely resemble the scattering/diffuse reflection and brightness observed in skeletal muscle as opposed to the specular reflection and stark, light/dark contrast observed in the solid walled honeycomb and rod designs. Investigation of the relationship between lattice design parameters and resulting echo intensity is needed to move toward clinically relevant muscle ultrasound phantoms from these proof-of-concept designs.

### Conclusion

The high-user dependency limiting the adoption need for muscle ultrasound phantoms to improve the validity and training methods of clinical and pre-clinical ultrasound applications. This would enable greater adoption of the imaging modality and enhance the quality of ultrasound assessments. SLA 3D printing with FormLabs' Elastic 50A resin was investigated as a method to mimic the anisotropic structure and imaging pattern of skeletal muscle. The acoustic properties of the resin combined with the 3D structure of the printed phantoms enabled replication of the hyper- and hypo-echoic pattern, while introduction of latticing created a structural control of the echo intensity of phantom images. This study serves as proof-of-concept that soft, 3D printed phantoms can replicate the characteristic muscle ultrasound imaging pattern with the ability to tune physiologically relevant echo intensity values via structural design.

### THE FOLLOWING REFERENCES ARE INCORPORATED BY REFERENCE HEREIN

1. Gans C. Fiber architecture and muscle function. Exerc Sport Sci Rev. 1982; 10:160-207.
2. Gans C, Bock W J. The functional significance of muscle architecture—a theoretical analysis. Ergeb Anat Entwicklungsgesch. 1965; 38:115-42.
3. Sacks R D, Roy R R. Architecture of the hind limb muscles of cats: functional significance. J Morphol. 1982; 173(2): 185-95.
4. Lieber R L, Friden J. Functional and clinical significance of skeletal muscle architecture. Muscle Nerve. 2000; 23(11):1647-66.
5. McGregor R A. It is not just muscle mass: a review of muscle quality, composition and metabolism during ageing as determinants of muscle function and mobility in later life. Longevity & Healthspan. 2014; 3.
6. Sipila S, Suominen H. Muscle ultrasonography and computed tomography in elderly trained and untrained women. Muscle Nerve. 1993; 16(3):294-300.
7. Goodpaster B H, Park S W, Harris T B, Kritchevsky S B, Nevitt M, Schwartz A V, et al. The loss of skeletal muscle strength, mass, and quality in older adults: the health, aging and body composition study. J Gerontol A Biol Sci Med Sci. 2006; 61(10):1059-64.
8. Addison O, Marcus R L, Lastayo P C, Ryan A S. Intermuscular fat: a review of the consequences and causes. Int J Endocrinol. 2014; 2014:309570.
9. Teigen L M, Kuchnia A J, Mourtzakis M, Earthman C P. The Use of Technology for Estimating Body Composition (Strengths and Weaknesses of Common Modalities in a Clinical Setting [Formula: see text]). Nutr Clin Pract. 2017; 32(1):20-9.
10. Manso Jimeno M, Vaughan J T, Geethanath S. Superconducting magnet designs and MRI accessibility: A review. Nmr Biomed. 2023: e4921.
11. Wald L L, McDaniel P C, Witzel T, Stockmann J P, Cooley C Z. Low-cost and portable MRI. J Magn Reson Imaging. 2020; 52(3): 686-96.
12. Frush D P, Applegate K. Computed tomography and radiation: understanding the issues. J Am Coll Radiol. 2004; 1(2):113-9.
13. Fletcher J G, Kofler J M, Coburn J A, Bruining D H, McCollough C H. Perspective on radiation risk in CT imaging. Abdom Imaging. 2013; 38(1):22-31.
14. Stock M S, Thompson B J. Echo intensity as an indicator of skeletal muscle quality: applications, methodology, and future directions. Eur J Appl Physiol. 2021; 121(2):369-80.
15. Campbell S E, Adler R, Sofka C M. Ultrasound of muscle abnormalities. Ultrasound Q. 2005;21(2):87-94; quiz 150, 3-4.
16. Smith J, Finnoff JT. Diagnostic and interventional musculoskeletal ultrasound: part 2. Clinical applications. P M R. 2009; 1(2):162-77.
17. Moreta M C, Fleet A, Reebye R, McKernan G, Berger M, Farag J, et al. Reliability and Validity of the Modified Heckmatt Scale in Evaluating Muscle Changes With Ultrasound in Spasticity. Arch Rehabil Res Clin Transl. 2020; 2(4):100071.
18. Alter K E, Karp B I. Ultrasound Guidance for Botulinum Neurotoxin Chemodenervation Procedures. Toxins (Basel). 2017;10(1).
19. Raithatha A, Ashraghi M R, Lord C, Limback-Stanic C, Viegas S, Amiras D. Ultrasound-guided muscle biopsy: a practical alternative for investigation of myopathy. Skeletal Radiol. 2020; 49(11):1855-9.
20. Picelli A, Bonetti P, Fontana C, Barausse M, Dambruoso F, Gajofatto F, et al. Is spastic muscle echo intensity related to the response to botulinum toxin type A in patients with stroke? A cohort study. Arch Phys Med Rehabil. 2012; 93(7):1253-8.
21. Fukumoto Y, Ikezoe T, Yamada Y, Tsukagoshi R, Nakamura M, Mori N, et al. Skeletal muscle quality assessed from echo intensity is associated with muscle strength of middle-aged and elderly persons. Eur J Appl Physiol. 2012; 112(4):1519-25.
22. Pillen S, Tak R O, Zwarts M J, Lammens M M, Verrijp K N, Arts I M, et al. Skeletal muscle ultrasound: correlation between fibrous tissue and echo intensity. Ultrasound Med Biol. 2009; 35(3):443-6.
23. Reimers K, Reimers C D, Wagner S, Paetzke I, Pongratz D E. Skeletal-Muscle Sonography-a Correlative Study of Echogenicity and Morphology. J Ultras Med. 1993; 12(2):73-7.
24. Arts I M P, Schelhaas H J, Verrijp K C N, Zwarts M J, Overeem S, van der Laak JAWM, et al. Intramuscular fibrous tissue determines muscle echo intensity in amyotrophic lateral sclerosis. Muscle & Nerve. 2012; 45(3):449-50.
25. Kwah L K, Pinto R Z, Diong J, Herbert R D. Reliability and validity of ultrasound measurements of muscle fascicle length and pennation in humans: a systematic review. J Appl Physiol (1985). 2013; 114(6):761-9.
26. George W. Deimel EJJMMH. Musculoskeletal Ultrasound in Physical Medicine and Rehabilitation. Current Physical Medicine and Rehabilitation Reports. 2013; 1:38-47.
27. Levent Özçakar T E, Gülümser Aydin, Kamal Mezian, Murat Kara, Eda Gürçay, Franco Franchignoni. Musculoskeletal ultrasound “threatening” physical and rehabilitation medicine: a prime caution regarding foxes. European Journal of Physical and Rehabilitation Medicine. 2017; 54(3):499-500.
28. Culjat M O, Goldenberg D, Tewari P, Singh R S. A Review of Tissue Substitutes for Ultrasound Imaging. Ultrasound in Medicine and Biology. 2010; 36(6):861-73.
29. Adkins A N, Dewald J P A, Garmirian L P, Nelson C M, Murray W M. Serial sarcomere number is substantially decreased within the paretic biceps brachii in individuals with chronic hemiparetic stroke. Proc Natl Acad Sci U S A. 2021; 118(26).
30. Slutsky D J, Hentz V R. Peripheral nerve surgery: practical applications in the upper extremity. Philadelphia: Churchill Livingstone Elsevier; 2006. xvi, 375 p. p.
31. Korthuis R J. Skeletal Muscle Circulation. Integrated Systems Physiology: from Molecule to Function to Disease. San Rafael (CA) 2011.
32. Feher J J. Quantitative human physiology: an introduction. Second edition. ed. Amsterdam; Boston: Elsevier/AP, Academic Press is an imprint of Elsevier; 2017. xiii, 968 pages p.
33. Huang C, Yao B, Li X, Li S, Zhou P. Muscle Fiber Diameter and Density Alterations after Stroke Examined by Single-Fiber EMG. Neural Plast. 2021; 2021:3045990.
34. Madsen E L, Zagzebski J A, Banjavie R A, Jutila R E. Tissue mimicking materials for ultrasound phantoms. Med Phys. 1978; 5(5):391-4.
35. Cafarelli A, Verbeni A, Poliziani A, Dario P, Menciassi A, Ricotti L. Tuning acoustic and mechanical properties of materials for ultrasound phantoms and smart substrates for cell cultures. Acta Biomater. 2017; 49:368-78.
36. Chen P, Pollet AMAO, Panfilova A, Zhou M, Turco S, den Toonder J M J, et al. Acoustic characterization of tissue-mimicking materials for ultrasound perfusion imaging research. Ultrasound in Medicine & Biology. 2022; 48(1):124-42.
37. Bilaniuk N, Wong G S K. Speed of Sound in Pure Water as a Function of Temperature. J Acoust Soc Am. 1993; 93(3):1609-12.
38. Delgross. Va, Mader C W. Speed of Sound in Pure Water. J Acoust Soc Am. 1972; 52(5):1442-&.
39. Zaidman C M, Malkus E C, Connolly A M. Muscle ultrasound quantifies disease progression over time in infants and young boys with duchenne muscular dystrophy. Muscle Nerve. 2015; 52(3):334-8.
40. ICRU. Tissue substitutes, phantoms, and computational modelling in medical ultrasound. Bethesda, Md.: International Commission on Radiation Units and Measurements; 1998. viii, 132 p. p.
41. Mast TD. Empirical relationships between acoustic parameters in human soft tissues. Acoustics Research Letters Online. 2000; 1(2):37-42.
42. Zell K, Sperl J I, Vogel M W, Niessner R, Haisch C. Acoustical properties of selected tissue phantom materials for ultrasound imaging. Phys Med Biol. 2007; 52(20):N475.
43. McGarry C K, Grattan L J, Ivory A M, Leek F, Liney G P, Liu Y, et al. Tissue mimicking materials for imaging and therapy phantoms: a review. Phys Med Biol. 2020; 65(23).
44. Damon B M, Buck A K, Ding Z. Diffusion-Tensor MRI Based Skeletal Muscle Fiber Tracking. Imaging Med. 2011; 3(6):675-87.
45. Damon B M, Froeling M, Buck A K W, Oudeman J, Ding Z H, Nederveen A J, et al. Skeletal muscle diffusion tensor-MRI fiber tracking: rationale, data acquisition and analysis methods, applications and future directions. Nmr Biomed. 2017; 30(3).
46. Lansdown D A, Ding Z H, Wadington M, Hornberger J L, Damon B M. Quantitative diffusion tensor MRI-based fiber tracking of human skeletal muscle. J Appl Physiol. 2007; 103(2):673-81.
47. Choi H F, Blemker S S. Skeletal Muscle Fascicle Arrangements Can Be Reconstructed Using a Laplacian Vector Field Simulation. Plos One. 2013; 8(10).

Although the foregoing invention has been described in some detail by way of illustration and example for purposes of clarity of understanding, certain changes and modifications may be practiced within the scope of the appended claims.

What is claimed is:

1. A phantom comprising:
a structure that, when subject to a B-mode ultrasound, is configured to generate return waves that mimic return waves produced by muscle tissue when subjected to B-mode ultrasound.

2. The phantom of claim 1, wherein the phantom has a longitudinal axis, wherein the structure comprises a first material, and wherein the structure defines a plurality of regions free of the first material along a first transverse axis perpendicular to the longitudinal axis.

3. The phantom of claim 2, wherein the structure comprises:
a plurality of rods that are elongated along the longitudinal axis; and
at least one support that spaces the plurality of rods along the first transverse axis, wherein the plurality of regions free of the first material are defined by spaces between the plurality of rods.

4. The phantom of claim 3, wherein the at least one support further spaces the plurality of rods along a second transverse axis that is perpendicular to the longitudinal axis and the first transverse axis.

5. The phantom of claim 3, wherein the plurality of rods are arranged in a honeycomb pattern.

6. The phantom of claim 3, wherein the plurality of rods have a diameter from about 1 mm to about 4 mm.

7. The phantom of claim 3, wherein the plurality of rods are cylindrical.

8. The phantom of claim 3, wherein the sequential rods of the plurality of rods are evenly spaced from each other.

9. The phantom of claim 2, wherein the structure comprises a body defining a plurality of longitudinally extending passages that define the plurality of regions free of the first material.

10. The phantom of claim 9, wherein the plurality of longitudinally extending passages are hexagonal in cross sections perpendicular to the longitudinal axis.

11. The phantom of claim 9, wherein the plurality of longitudinally extending passages have an inscribed diameter from about 1 mm to about 4 mm.

12. The phantom of claim 9, wherein the structure comprises walls defining the longitudinally extending passages, wherein the structure defines transverse openings extending through the walls of the longitudinally extending passages.

13. The phantom of claim 2, wherein the plurality of regions free of the first material are full of a gas or liquid.

14. The phantom of claim 2, wherein the plurality of regions free of the first material are full of a second solid material that is different from the first material.

15. The phantom of claim 1, wherein the phantom does not have an internal structure that physically resembles muscle.

16. The phantom of claim 1, wherein the phantom has a length from 20 mm to 100 mm.

17. The phantom of claim 2, wherein the first material comprises photocured elastic resin.

18. The phantom of claim 2, wherein the first material has a hardness from about 40 to about 60 on the shore A durometer scale.

19. A method comprising:
forming the phantom of claim 1 by 3D printing.

20. A method comprising:
immersing the phantom of claim 1 in water;
subjecting the phantom to a B-mode ultrasound; and
capturing return waves generated by the phantom.
